(19) United States
(12) Patent Application Publication
(10) Pub. No.: US 2008/0243181 A1
(43) Pub. Date: Oct. 2, 2008

(54) Touch-Switched Luminous Plastic Tweezers

(75) Inventors: Ralph Schneider, Monheim (DE); Udo Guntermann, Krefeld (DE)

Correspondence Address: NORRIS, MCLAUGHLIN & MARCUS, PA, 875 THIRD AVENUE, 18TH FLOOR, NEW YORK, NY, 10022, US

(73) Assignee: BAYER MATERIALSCIENCE AG, Leverkusen (DE)

(21) Appl. No.: 12/090,123

(22) PCT Filed: Oct. 9, 2006
(86) PCT No.: PCT/EP06/09724

§ 371 Date: Apr. 14, 2008

(30) Foreign Application Priority Data
Oct. 15, 2005 (DE) 10 2005 049 446.3

(51) Int. Cl. A61B 17/50; F21V 33/00

(52) U.S. Cl. 606/211; 362/109; 362/572; 606/131

(57) ABSTRACT

Luminous tweezers are described having two tweezer branches (3), at the ends of which gripping/illuminating jaws (9) are provided, and at least one light source (5) with a switchable electricity supply (6; 7), the tweezer branches (3) consisting at least partially of transparent plastic and forming a light guide for the light from the light source, characterised in that the light source (5) can be switched by an electrical touch circuit (8; 1; 10).

The present invention relates to a pair of tweezers according to the precharacterising clause of Patent Claim 1.

Objects with small dimensions are conventionally handled using tweezers. In order to be able to perform this task under poor lighting conditions, additional illumination is directed onto the objects to be handled. The light source may in this case be fitted in the structure of the tweezers. In the various embodiments, the light from a light source positioned in the longitudinal axis of the tweezers, and close to the fork point of the tweezer branches, is generally directed onto the gripping tip (cf. EP 70 459 A1 or DE 102 40 490 A1). Either this is done on the direct light path or, after the light has been injected, the light is transported by using clear polymers from the fork point of the tweezers to the gripping tips, where it is output.

To this end, for the light guiding, an optical waveguide may be fitted on/in the tweezer branches (Patent JP10-151573,A), or the tweezer material simultaneously fulfils the functions of light guiding and handling small objects (JP10-165409,A). In these described cases, after the actuation of a mechanical switch, the light is transported via a coupled/applied portable light generator from the fork point of the tweezers to the gripping tip of the tweezers, with the aid of the light-guiding properties of clear plastics. In the described case, this is very elaborate and involved, as well as ineffective. In many cases, it is difficult for the light from the illuminating instrument to be directed by this type of light transport onto the objects to be handled, since the fork point is a disadvantageous position in design terms for the light injection and represents the most distant position from the gripping tip, i.e. the place where the light is output. A very great deal of light is lost owing to the attenuating properties of the polymer being used and/or the light may potentially escape unintentionally at many positions.

The switch mechanisms, which are usually complicated or elaborate in various regards, represent a potential source of error and also have a limited functional number of cycles and therefore lifetime. Furthermore, they are usually very complicated to handle. In the least favourable case, attempting to activate the switch requires use of the second hand and therefore interruption of its own activity. Fitting latched switches leads to wastage of energy, and timer switching mechanisms unnecessarily increase the complexity.

Design separation of the light generator unit and the plastic gripping tool (cf. JP10-165409,A) leads to a poor distribution of the weight ratios in relation to the centre of mass, which is a great impediment to fulfilling the function of handling small objects.

Using polymers as a plastic material leads to the risk of static charging. This is a risk due to which sensitive objects may be damaged or destroyed by sudden discharging of the static charge.

It is an object of the invention to provide a device which allows simple handling of objects with a small size even under unfavourable lighting conditions, minimises the risk of a possible static discharge when using polymers as a design material for light guiding and tweezer-like gripping functions, and eliminates the source of error and the handling disadvantage due to mechanical switching mechanisms. In order to optimise the combined functional requirement for illumination and simultaneous gripping of small objects, a combined illuminating and gripping structure is established for the gripping jaws of the tweezers.

The object is achieved by the features specified in the characterising part of Patent Claim 1.

The tweezers according to the invention comprise two tweezer branches having first ends, i.e. the gripping/illuminating jaws, which are used for handling small objects and at the same time specifically directed as well as partially undirected output of the light injected into the tweezer branches, in order to illuminate the object to be gripped and the immediate vicinity for accurate orientation.

The invention provides luminous tweezers having two tweezer branches, at the ends of which gripping/illuminating jaws are provided, and at least one light source with a switchable electricity supply, characterised in that the light source/s can be switched by an electrical touch circuit. The tweezer branches consist at least partially of transparent plastic and form a light guide for the light from the light source.

A preferred form of the luminous tweezers is characterised in that the touch circuit comprises at least two touch zones made of electrically conductive plastic, in particular transparent electrically conductive plastic.

The electrical touch circuit is preferably based on a Darlington circuit.

The polymer of the electrically conductive plastic for the touch zones is preferably selected from the group of polythiophenes, polyanilines or polypyrroles. Polyalkylene dioxythiophene is particularly preferred, and poly(3,4-ethylene dioxythiophene) is more particularly preferred.

A preferred variant of the luminous tweezers is characterised in that the light from the luminous means is guided through the tweezer branches to the gripping/illuminating jaws and is output on the inside there via the gripping/illuminating jaws.

The luminous means preferably comprises a light-emitting diode or laser diode. The electricity supply preferably comprises an accumulator or a battery.

The transparent plastic of the tweezer branches is preferably based on polycarbonate, polyester, polyacrylate, particularly preferably polycarbonate or PMMA.

In a particularly preferred form of the luminous tweezers, the touch zones are applied on both tweezer branches. The light is switched on by touching both zones with the hand.

The light is injected at a position, between the two ends of each branch, which is suitable in terms of design and optics. The branches are resiliently connected together by a material, form of force fit, and thus fulfil their tweezer-like gripping function. The electrical components for generating the required light are spatially positioned or integrated between and/or in the respective tweezer branches.

A mechanical switch for switching the light source on/off is obviated by application, technically configured in a wide variety of ways, of a layer of electrically conductive intrinsic polymer. This layer here fulfils the function both of an antistatic and that of the contact/switching surfaces of an electrical touch circuit, achieved by spatially separating/interrupting the coating between the tweezer branches which therefore form the two required separate contact surfaces. The touch circuit is advantageous since illumination of the tweezers is triggered only by touch and therefore only during use. Consequently, the circuit saves on electricity and prevents premature or inadvertent discharging of the batteries/accumulators. Mechanical moving parts, and therefore sources of wear and error, are furthermore eliminated.

The invention furthermore provides the use of the luminous tweezers as a medical instrument, in particular as tick removers.

### EXAMPLE

The invention will be explained in more detail with the aid of schematic drawings, in which:

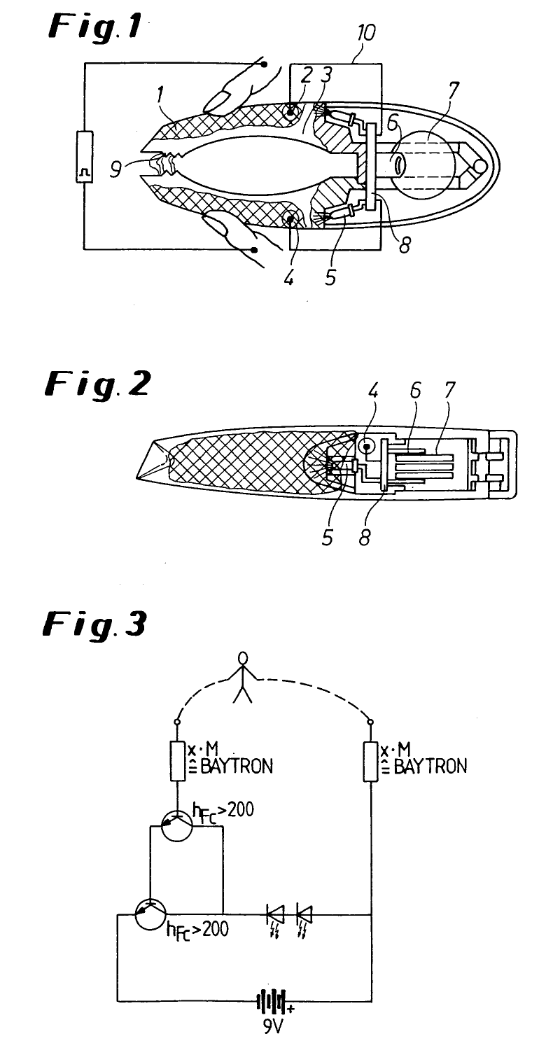

FIG. 1 shows a front view of a pair of tweezers with integrated illumination according to the invention, having an electrical touch circuit using an intrinsic polymer as an antistatic and a switching contact surface according to a first embodiment of the invention.

FIG. 2 shows the side view of the tweezers in FIG. 1.

FIG. 3 shows the circuit principle for the tweezers.

As shown in FIG. 1, a pair of luminous tweezers comprises two resiliently connected tweezer branches 3, which consist of a clear polymer (polycarbonate) to fulfil the light guiding and an upper part made of a polymer, ceramic or metal to fulfil the gripping function. At the lower end, the tweezer branches are separated from one another in order to hold an object which has small dimensions.

The small objects to be gripped are illuminated by injecting the light, generated in the light-emitting diodes 5, via a defined surface into the transparent body of the tweezer branches, the light then being output in the specifically configured gripping/illuminating region 9 in the direction of the object to be gripped.

In the right-hand region of the luminous tweezers with a touch circuit, the electrical components of the illumination instrument 1, 4, 5, 6, 7, 8 are positioned partly in the respective tweezer branches and partly between the respective tweezer branches.

The illumination system with a touch circuit using an antistatic as contact surfaces of an electrical touch circuit consists of one or more light sources 5 (LEDs), and an electricity store (battery, accumulator or capacitor) 7 which is connected via a frame 6 to a supporting unit for the electronic components (printed circuit board, foil) 8, to which the light sources 5 are also connected. The tweezer branches are respectively provided with an electrically conductive polymeric coating 1 in a subregion. Between the coating 1 and the switching/secondary current loop of the electrical circuit of the touch unit, there is a force, form or material fit connection 4, here, in a first embodiment, a material fit 4 with the electrical line 10 using electrically conductive adhesive. In order to switch the luminous means 5 on, a connection is established 2 between the two tweezer branches, preferably by the user's fingers or hand regions during use of the tweezer-like gripping tool.

FIG. 3 shows a schematic representation of a possible embodiment of such an electrical touch circuit using an antistatic or electrically conductive coating, based on an intrinsic polymer, for luminous tweezers. The construction of the circuit from a primary current loop and a secondary current loop, which are connected by an energy-sensitive circuit, is characteristic in this case. The primary current loop supplies the light source(s) with energy, while the secondary/control current loop together with an energy-sensitive circuit, here, in a first embodiment, established by a transistor arrangement of the so-called Darlington circuit, represents the controller or switching mechanism of the primary circuit. The structure of the touch circuit embodiment shown here in the secondary current loop consists of the Darlington arrangement, on the first base of which there is one of two surfaces of the intrinsic polymer contact surface, which are spatially separated from one another. This contact surface is applied onto one of the two tweezer branches. It is transparent in its appearance and simultaneously fulfils the function of an antistatic. The second intrinsic contact surface is located on the second tweezer branch, is configured identically to the first (FIG. 1) 1 and is connected to the positive terminal of the energy source. Via a physical connection additionally to be established between the contact surfaces, the control current loop is closed, the transistors are turned on and the primary current loop is therefore switched on. After the physical connection is interrupted, the transistor circuit also immediately interrupts the primary current loop and therefore switches the light sources 5 off.

The coating consists of an electrically conductive polyethylene dioxythiophene/polystyrene sulfonic acid layer.

1. A luminous tweezers comprising
two tweezer branches including ends formed as gripping/illuminating jaws and at least one light source with a switchable electricity supply, the tweezer branches formed partially from transparent plastic and including a light guide for the light from the light source, wherein the light source is capable of being switched by an electrical touch circuit.

2. The luminous tweezers according to claim 1, wherein the touch circuit comprises at least two touch zones made of electrically conductive plastic.

3. The luminous tweezers according to claim 1, wherein the electrical touch circuit is based on a Darlington circuit.

4. The luminous tweezers according to claim 2, wherein the electrically conductive plastic includes a polymer and wherein the polymer is selected from the group of polythiophenes, polyanilines or polypyrroles, preferably polyalkylene dioxythiophene, particularly preferably poly(3,4-ethylene dioxythiophene).

5. The luminous tweezers according to claim 1, wherein the light from the luminous means is guided through the tweezer branches to the gripping/illuminating jaws and is output on the inside via the gripping/illuminating jaws.

6. The luminous tweezers according to claim 1, wherein the luminous means comprises a light-emitting diode or laser diode and/or the electricity supply comprises an accumulator or a battery.

7. The luminous Luminous tweezers according to claim 1, wherein the transparent plastic of the tweezer branches is based on polycarbonate.

8. The luminous Luminous tweezers according to claim 2, wherein the touch zones are applied on both tweezer branches.

9. The luminous Luminous tweezers according to claim 2, wherein the touch zones (1) have antistatic properties.

10. A method of using the luminous tweezers according to claim 1 as a medical instrument.

11. The luminous tweezers according to claim 4, wherein the electrically conductive plastic is a transparent electrically conductive plastic.

12. The luminous tweezers according to claim 2, wherein the polymer is polyalkylene dioxythiophene. particularly preferably poly(3,4-ethylene dioxythiophene).

13. The luminous tweezers according to claim 2, wherein the polymer is poly(3,4-ethylene dioxythiophene).

14. A method of using the luminous tweezers according to claim 10, wherein the medical instrument is a tock remover.
